(19) United States
(12) Patent Application Publication
(10) Pub. No.: US 2007/0298032 A1
(43) Pub. Date: Dec. 27, 2007

(54) Proteins Involved In Quorum Sensing

(75) Inventors: Gary Robinson, Canterbury (GB); Hannah Rising, Broomfield (GB)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(73) Assignee: UNIVERSITY OF KENT, Canterbury (GB)

(21) Appl. No.: 10/577,124

(22) PCT Filed: Nov. 10, 2004
(86) PCT No.: PCT/GB04/04775

§ 371 Date: May 1, 2007

(30) Foreign Application Priority Data
Nov. 10, 2003 (GB) 0326192.2

(51) Int. Cl. A61K 38/16; A61K 31/7088; A61K 39/02; A61K 39/395; A61P 31/04; C07K 16/12; G01N 33/569

(52) U.S. Cl. 424/131.1; 424/150.1; 424/164.1; 424/234.1; 435/7.32; 514/2; 514/44; 530/387.2; 530/387.9

(57) ABSTRACT

The invention relates to a method of regulating quorum sensing in bacteria by modulating the activation of LuxR or a homologue thereof. Such modulation generally occurs when the bacteria are in the pre-quorate or quorate stage and may be achieved by targeting antibodies to LuxR or a homologue thereof.

All documents cited herein are incorporated by reference in their entirety.

### TECHNICAL FIELD

This invention is in the field of signal transduction in bacteria. More particularly, the invention relates to the mechanism of quorum sensing in bacteria and control of this mechanism by antibodies and vaccines.

### BACKGROUND ART

Quorum sensing is a phenomenon that was first termed in 1994 by Fuqua et al. (J. Bacteriology, 176:269-276). However, the phenomenon of “autoinduction” in the bioluminescent organism Photobacteria fischeri (later to become Vibrio fischeri) which underpinned the development of quorum sensing research was first described in 1970 by Nealson, Platt and Hastings (J. Bacteriol. 104(1):313-22). Whilst working on the physiology of luminescence of Photobacteria fischeri (Vibrio fischeri), they noticed that there was no appreciable amount of luminescence emitted by the bacteria until the population of cells had reached a concentrated culture. This phenomenon was also noticed in conjunction with the squid Euprymna scolopes, where the bacteria colonise the squid's light organ to a concentration of 10¹⁰ to 10¹¹ cells/ml, causing the organ to glow. However, when present in diffuse amounts in seawater, no bioluminescence is noticed. It therefore seemed that the population of bacteria could sense its own concentration and either initiate the bioluminescent pathway or remain unlit. This mechanism which combines cell-cell communication and cell density is defined as quorum sensing.

In V. fischeri the system is controlled by the lux operon which consists of a number of genes including luxI, encoding the autoinducer synthase, and luxR which encodes an autoinducer-dependent activator of the luminescence genes (Sitnikov et al. 1995, Mol. Microbiol.17:801-812). The autoinducer signal molecule, which in many Gram negative bacteria is an N-acylated homoserine lactone (AHL), is excreted by each cell into the surrounding media. This autoinducer is then taken up by other cells and binds the LuxR receptor protein which then activates the lux operon, causing the production of luciferase and therefore bioluminescence. Kolibachuk and Greenberg (J. Bacteriol. 1993, 175(22):7307-7312) proposed a model for the signal transduction pathway that is used in this process. Their experiments suggested that LuxR is found on the inner surface of the bacterial cytoplasmic membrane. Further evidence of this is given in “Transcriptional Activation by LuxR” (Stevens and Greenberg, 1999, in “Cell-cell signalling in bacteria” Eds. Dunny & Winams).

The phenomenon of quorum sensing is not just limited to V. fischeri, but has been described in a lumber of Gram-negative and Gram-positive bacteria Many of these bacteria use quorum sensing to control the production of virulence determinants thus allowing the pathogenic bacteria to avoid host defences until a sufficient population has been reached. Examples of pathogens that utilise quorum sensing include Bacillus subtilis, Streptococcus pneumoniae, Enterococcus faecalis, Pseudomonas aeruginosa, Burkholderia cepacia, Yersinia pestis, Yersinia enterocolitica and Yersinia pseudotuberculosis (Cámara et al. 2002 The Lancet Infectious Diseases 2:667-676). Pseudomonas fluorescens is thought to play an important role in Crohn's disease and it is further believed that its quorum sensing signalling molecule plays a role in immune modulation. With the emergence of organisms that are resistant to many antibiotics such as methicillin-resistant Staphylococcus aureus (MRSA), quorum sensing represents a novel therapeutic target offering the opportunity to attenuate virulence, thus controlling infection by blocking cell-cell communication.

Biofilms form when bacteria adhere to surfaces in aqueous environments and begin to excrete a slimy, glue-like substance that can anchor them to all kinds of material—such as metals, plastics, soil particles, medical implant materials, and tissue. The production of this “slimy, glue-like substance” and other virulence determinants are thought to be controlled by quorum sensing (Latifi et al. 1995, Mol. Microbiol. 17:333-344). A biofilm can be formed by a single bacterial species, but more often biofilms consist of many species of bacteria, as well as fungi, algae, protozoa, debris and corrosion products. Essentially, biofilms may form on any surface exposed to bacteria and some amount of water (Costerton et al. 1987, Ann. Rev. Microbiol. 41:435-464). Once anchored to a surface, biofilm microorganisms carry out a variety of detrimental or beneficial reactions (by human standards), depending on the surrounding environmental conditions. Microbial biofilms on surfaces cause billions of dollars yearly of equipment damage, product contamination, energy losses and medical infections.

### DISCLOSURE OF THE INVENTION

The present invention is based on the discovery that the protein complexes involved in quorum sensing are found on the outer surface of the bacterial membrane during certain stages of growth. In particular, it has been found that the protein LuxR is an integral part of a signal transduction complex that is found on the outer surface of the V. fischeri bacterial membrane. The interaction of the protein LuxR and homologues thereof with the signal molecule (which for V. fischeri is an N-acylated homoserine lactone, specifically N-3-(oxohexanoyl)homoserine lactone) is therefore a target for modulation to control quorum sensing, attenuate virulence and control bacterial growth and/or infection. The finding that the protein LuxR forms part of a signal transduction complex on the outer surface of the bacterial membrane facilitates extracellular modulation of the activation of LuxR or homologue of LuxR. It therefore facilitates modulation of the activation of LuxR or a homologue of LuxR by a ligand, such as an antibody, which is unable to cross the bacterial cell membrane. Furthermore, other bacteria that use quorum sensing have similar signal transduction complexes comprising a homologue of LuxR. Two such homologues are the LasR protein of P. aeruginosa and the SdiA protein of E.coli.

In a first aspect, the invention provides a method of regulating quorum sensing in bacteria comprising modulating the activation by a signalling molecule of LuxR or a homologue thereof. Preferably the modulation is extracellular.

The present invention may be employed in the regulation of any bacteria employing quorum sensing. Proliferation of such bacteria involve distinct stages, namely, pre-quorate, quorate and post-quorate. For modulation, preferably the bacteria are in the pre-quorate or quorate stage, more preferably they are in the pre-quorate stage.

The activation of LuxR or a homologue of LuxR may be upregulated or downregulated. Preferably the activation of LuxR or a homologue of LuxR is downregulated and more preferably is prevented.

Homologues of LuxR are proteins that share a common evolutionary ancestor with LuxR (as described by Gray, K. M. & Garey, J. R., 2001, Microbiology, 147:2379-2387) and are induced in quorum sensing. Homologues of LuxR are described in PROSITE as being members of the LuxR family of proteins (see http://us.expasy.org/cgi-bin/nicedoc.pl?PS00622). Preferably they are proteins that are found on the outer surface of bacterial membranes during the pre-quorate and quorate phases of bacterial growth and which bind a signalling molecule and are then able to activate transcription. Preferably, they are proteins that share sequence identity with LuxR. Preferably homologues of LuxR share more than 40% sequence identity with LuxR (e.g. more than 50%, 60%, 70%, 80%, 90%, 95%, 99% or more). Preferably homologues of LuxR have residues corresponding to those of LuxR. Preferably homologues of LuxR have residues corresponding to the following residues of LuxR when aligned using the Clustal alignment algorithm (Higgins, D. G., Bleasby, A. J. and Fuchs, R., 1992, CLUSTAL V: improved software for multiple sequence alignment. Computer Applications in the Biosciences (CABIOS), 8(2):189-191): TRP66, TYR70, ASP79, PRO80, GLY121, GLU187 and GLY197. Preferably the homologue of LuxR is selected from the group consisting of AhlR, AhyR, AsaR, BafR, BisR, BpsR, BviR, CarR, CepR, CerR, CinR, CsaR, CviR, EagR, EcbR, EchR, EsaR, ExpR, HalR, LasR, LuxS, M118752, MupR, PcoR, PhzR, PmlR, PpuR, PsmR, PsyR, RaiR, RhiR, RhlR, SdiA, SdiR, SmaR, SolR, SpnR, SprR, SwrR, TraR, TriR, TrlR, TrnR, VanR, VsmR, Y4qH, YenR, YpeR, YpsR, YruR, YtbR and YukR.

The homologue of LuxR may be a positive regulator (like LuxR) and upregulate expression of certain proteins at high cell density, or may be a negative regulator and upregulate expression expression of certain proteins at low cell density. Examples of negative regulators include SdiA and EsaR (von Bodman et al. 1998 Proc Natl Acad Sci 95(13):7687-92). SdiA homologues are highly conserved, as is demonstrated in Table 1, which shows the output from the Swiss Prot/TrEMBL database (see http://bo.expasy.org/sprot/) when queried with SdiA.

TABLE 1

| Sequence | Organism | Identity |
|---|---|---|
| P07026 | E. coli | 100% |
| Q8XBD0 | E. coli 0157: H7 | 99% |
| Q7AD11 | E. coli 0157: H7 | 99% |
| Q8FGM5 | E. coli O6 | 97% |
| Q83R45 | Shigella flexneri | 96% |
| Q7UAB1 | Shigella flexneri | 96% |
| Q8Z5T1 | Salmonella typhi | 72% |
| Q7CQB6 | Salmonella typhimurium | 71% |
| O66040 | Salmonella typhimurium | 71% |

SdiA is, amongst other functions, able to regulate expression of the AcrAB multidrug efflux pump (Rahmati et al. 2002 Molecular Microbiology 43:677-685). Therefore if the signal transduction complex comprising SdiA is blocked, then expression of the AcrAB multidrug efflux pump is downregulated and the bacterial cell comprising the gene encoding that pump is sensitised to antibiotics.

LuxR and the homologues of LuxR use an N-acylated homoserine lactone as the signalling molecule.

Preferably the activation of LuxR or a homologue of LuxR is modulated by providing a ligand capable of binding to LuxR or a homologue of LuxR and thereby blocking activation by the signalling molecule. Preferably, the ligand is an antibody. Most preferably, said antibody is a monoclonal antibody.

By “activation of LuxR or a homologue of LuxR”, we mean that LuxR or a homologue is bound by a signalling molecule and is therefore able to activate transcription.

The ligand of the present invention preferably immunoreacts with LuxR or a homologue of LuxR between the negative regulation domain and the autoinducer-binding domain. In the case of LuxR, which is a polypeptide of 250 amino acids, the ligand preferably binds between amino acid residues 19 and 80, more preferably between amino acid residues 19 and 31. More preferably, the ligand employed in the invention immunoreacts with the amino acid sequence TCNNNKDINQC.

The specific sequence which was used for the production of ligands was derived from V. fischeri strain ES114.

The method of the invention also includes the use of antibodies that immunoreact with those ligands which immunoreact with LuxR or a homologue of LuxR. These anti-idiotypic antibodies may then be used to sequester the signalling molecules that activate LuxR or homologues of LuxR. Preferably the anti-idiotypic antibodies of the invention immunoreact with N-acylated homoserine lactones.

Ligands used in the invention may be conjugated to a detectable label. Preferred labels include radioisotopes, fluorescent labels, heavy metal molecules and enzymes. According to a further aspect, the invention provides methods of detecting quorum sensing bacteria comprising use of such labelled ligands.

The invention also includes pharmaceutical compositions comprising LuxR, a homologue of LuxR, a fragment of LuxR, a fragment of a homologue of LuxR or a nucleic acid encoding one of these polypeptides, or a ligand capable of binding one of these polypeptides. The polypeptides, fragments thereof or nucleic acid encoding one of these polypeptides may induce an immunogenic response in a host. Pharmaceutical compositions containing antibodies that immunoreact with LuxR or homologues of LuxR can be used to bind to bacteria, thus labelling them for attack by a host's immune defences.

Vaccine compositions are also contemplated that comprise LuxR, a homologue of LuxR, a fragment of LuxR, a fragment of a homologue of LuxR or a nucleic acid encoding one of these polypeptides or a quorum sensing signalling molecule. LuxR, a homologue of LuxR, a fragment of LuxR, a fragment of a homologue of LuxR or a nucleic acid encoding one of these polypeptides or a quorum sensing signalling molecule may be used to induce an immunogenic response in a host. Such responses will result in the production of antibodies and will prime the host's immune system against bacterial infection.

The invention also provides the use of LuxR, a homologue of LuxR, a fragment of LuxR or a homologue of LuxR, a nucleic acid encoding one of these polypeptides, a ligand, a pharmaceutical composition or a vaccine composition for use in therapy. Furthermore the invention provides the use of LuxR, a homologue of LuxR, a fragment of LuxR or a homologue of LuxR, a nucleic acid encoding one of these polypeptides, an antibody, a pharmaceutical composition or a vaccine composition according to the invention in the manufacture of a medicament for the treatment of a disease in which quorum sensing is implicated.

The invention also provides the use of LuxR, a homologue of LuxR, a fragment of LuxR or a homologue of LuxR, a nucleic acid encoding one of these polypeptides, a ligand, a pharmaceutical composition or a vaccine composition for sensitising an antibiotic resistant bacterium to an antibiotic.

The invention also provides the use of LuxR, a homologue of LuxR, a fragment of LuxR or a homologue of LuxR, a nucleic acid encoding one of these polypeptides, a ligand, a pharmaceutical composition or a vaccine composition in the manufacture of a medicament for sensitising an antibiotic resistant bacterium to an antibiotic.

By “sensitising”, we mean that a bacterium previously resistant to a given antibiotic is rendered sensitive to that antibiotic such that its growth or reproduction is inhibited by that antibiotic or it is killed by that antibiotic. Preferably the sensitisation of the bacterium is achieved by decreasing or preventing expression of an efflux pump such as the AcrAB multidrug efflux pump.

The invention also provides the use of LuxR, a homologue of LuxR, a fragment of LuxR or a homologue of LuxR, a nucleic acid encoding one of these polypeptides, a ligand, a pharmaceutical composition or a vaccine composition in the manufacture of a medicament for the treatment of a disease in which quorum sensing is implicated wherein the patient suffering from that disease is refractive to antibiotic therapy alone.

By “refractive” we mean that prior to treatment with the medicament according to the invention the subject is a hypo-responder or does not respond to antibiotic therapy.

The invention also provides the use of LuxR, a homologue of LuxR, a fragment of LuxR or a homologue of LuxR, a nucleic acid encoding one of these polypeptides, a ligand, a pharmaceutical composition or a vaccine composition in the manufacture of a medicament for the treatment of a disease in which quorum sensing is implicated wherein the medicament is administered in conjunction with an antibiotic.

The invention also provides the use of an antibiotic in the manufacture of a medicament for the treatment of a disease in which quorum sensing is implicated wherein the subject being treated is pre-administered with a pharmaceutical composition or vaccine according to the invention.

Such diseases include those caused by the bacteria Bacillus subtilis, Streptococcus pneumoniae, Staphylococcus aureas, Vibrio salmonicida, Aeromonas hydrophila, Burkhoderia ambifaria, Burkholderia pseudomallei, Burkholderia mallei, Burkholderia stabilis, Burkholderia vietnamiensis, Burkholderia multivorans, Escherichia coli, Serratia marcescens, Salmonella typhi, Brucella suis, Brucella melitensis, Yersinia ruckeri, Hafina alvei, Shigella flexneri, Serratia liquefaciens, Enterococcus faecalis, Pseudomonas aeruginosa, Burkholderia cepacia, Pseudomonas fluorescens, Providencia stuartii, Klebsiella aerogenes, Yersinia pestis, Yersinia enterocolitica or Yersinia pseudotuberculosis.

The diseases include Crohn's disease and Cystic Fibrosis, cellulites and ecthyma, Glanders, melioidosis, meningitis, septicaemia, pneumonia, enteric infections and urinary tract infections, food poisoning, chest infections, typhoid fever, Malta disease, blood stream infections, shigellosis, salmonelliosis, black death and gastroenteritis, hitra disease in Atlantic salmon, haemmorrhagic septicaemia in marine fish, spontaneous abortion in pigs and sheep, red mouth disease in rainbow trout, and cranial and eye lesions in fish.

Antibiotics for use as described above include erythromycin A, rifampin, tetracycline, chloramphenicol, norfloxacin, nalidixic acid and penicillin G. Preferred antibiotics are those that are pumped out of antibiotic resistant bacterial cells by an efflux pump. One such efflux pump is AcrAB which has been shown to increase bacterial resistance to the antimicrobial agents sodium dodecyl sulphate, sodium deoxychlorate, sodium chlorate, Triton X-100, crystal violet, acriflavine, fusidic acid, novobiocin, erythromycin A, rifampin, tetracycline, chloramphenicol, norfloxacin, nalidixic acid and penicillin G (see Nikaido et al. 1998 J. Bacteriology 180(17):4686-4692). Particularly preferred antibiotics for administration in the uses described above include tetracycline and nalidixic acid. An antibiotic may be administered by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Preferably an antibiotic is administered in a dose of 0.01-1000 mg/kg, more preferably 10-500 mg/kg, more preferably 50-400 mg/kg, more preferably 100-200 mg/kg. These doses may be administered hourly, daily or weekly.

The invention also provides a kit comprising for simultaneous, separate or sequential use (i) LuxR, a homologue of LuxR, a fragment of LuxR or a homologue of LuxR, a nucleic acid encoding one of these polypeptides, an antibody, a pharmaceutical composition or a vaccine composition according to the invention; and (ii) an antibiotic.

The methods of the present invention may be used to inhibit bacteria. As used herein, inhibition of bacteria means the prevention of phenotypic change. Accordingly, the present invention provides a method of inhibiting bacteria comprising modulating the activation by a signalling molecule of LuxR or a homologue thereof.

The methods of the invention may also be used to inhibit biofilms. As used herein, inhibition of a biofilm includes prevention of biofilm formation and growth, and destruction of biofilms. Accordingly, the present invention provides a method of inhibiting a biofilm comprising modulating the activation by a signalling molecule of LuxR or a homologue thereof.

The invention also provides a method of decreasing the minimum inhibitory concentration (MIC) value for antibiotics for a given bacterial strain (i.e. the lowest concentration of a particular antibiotic which can inhibit the growth of a given strain of bacteria) or minimum bactericidal concentration (MBC) value (i.e. the lowest concentration which can kill a given strain of bacteria). The method preferably comprises contacting a bacterium with LuxR, a homologue of LuxR, a fragment of LuxR or a homologue of LuxR, a nucleic acid encoding one of these polypeptides, an antibody, a pharmaceutical composition or a vaccine composition according to the invention. The MIC or MBC may be detected by an antibiotic susceptibility test known in the art, such as the disc agar diffusion test.

The invention also provides a method of detection of quorum sensing bacteria comprising;
(i) probing a sample of bacteria with a labelled ligand according to the invention, and
(ii) detecting the presence of ligand attached to bacteria.

In an alternative embodiment the method of detection of quorum sensing bacteria comprises;
(i) probing a sample of bacteria with a first ligand that binds to LuxR or a homologue of LuxR,
(ii) probing said first ligand with a second, labelled ligand, and
(iii) detecting the presence of the second antibody attached to bacteria.

Preferably the ligands used in the methods of detection are antibodies. More preferably they are monoclonal antibodies.

The invention also provides a method of detecting antibodies specific for LuxR or a homologue thereof comprising:
(i) probing a sample of serum with whole bacterial cells expressing whole or a fragment of LuxR or a homologue thereof,
(ii) probing the bacteria/antibody complex with a second, labelled antibody, and
(iii) detecting the presence of the second antibody attached to the bacteria/first antibody complex.

In an alternative embodiment, the method of detecting antibodies specific for LuxR or a homologue of LuxR comprises:
(i) probing a sample of serum with purified LuxR or a fragment or homologue thereof,
(ii) probing the bacterial protein/antibody complex with a second, labelled antibody, and
(iii) detecting the presence of the second antibody attached to the bacteria/first antibody complex.

Quorum sensing relies on signalling molecules to activate transcription. A variety of molecules are used as signalling molecules in quorum sensing as mentioned in Table 2. These ligands bind to their receptor which, for LuxR and its homologues, is found on the outer membrane of bacteria during the pre-quorate growth phase. This then causes the initiation of transcription after migration into the cytosol.

Ligands may be produced that immunoreact with LuxR, homologues of LuxR or fragments of these proteins. These ligands prevent the quorum sensing signalling molecules from binding to LuxR or its homologues, thus disrupting quorum sensing. This disruption can therefore prevent transcriptional activation and so prevent phenotypic changes associated with quorum sensing. Examples of phenotypic changes that can be prevented include the production of luciferase mediated by LuxR and the production of virulence determinants mediated by LasR. Such downregulation of virulence determinant production by pathogenic bacteria thus provides an effective means of therapy. Accordingly the present invention provides a method of downregulating the production of virulence determinants comprising downregulating the activation by a signalling molecule of a homologue of LuxR.

LasR is a homologue of LuxR and is a protein involved in virulence factor production in Pseudomonas aeruginosa. This organism has both cell-associated (flagella, pili and non-pilus adhesins, alginate/biofilm forming properties and lipopolysaccharide) and extracellular virulence factors (proteases, hemolysins, exotoxin A, exoenzyme S and pyocyanin). Ps. aeruginosa is a cause of infection in Cystic Fibrosis, burns victims and those with immunodeficiencies. This makes it a desired target for therapy. Many strains are also resistant to a number of antibiotics.

A further homologue of LuxR is SdiA. SdiA has been shown to be a negative regulator of the expression of EspD and Intimin in enterhaemorrhagic Escherichia coli (EHEC) O157:H7 (Kanamaru et al. Mol Micro 2000, 38:805-816).

Pseudomonas fluorescens has been shown to play a role in Crohn's disease. As well as causing infection in this disease state, it also encodes a T cell superantigen that results in autoimmunity, thus exacerbating disease (B. Wei et al. Infect Immun. 2002, 70(12):6567-75). It is known that Ps. fluorescens has homologues of LuxR. It is therefore likely that these homologues of LuxR play a role in the production of virulence determinants important in Crohn's disease. Inhibition of this signalling system is therefore likely to provide an effective method of therapy for the treatment of Crohn's disease by downregulating the production of said virulence determinants.

The methods of the invention may be applied to both Gram negative and Gram positive bacteria. Preferably the method of the invention is applied to Gram negative bacteria, preferably those which utilise homoserine lactones (HSLs) as the signal molecule, and more preferably those that utilise N-acylated homoserine lactone as the signalling molecule. As detailed in Table 2, there are a wide variety of bacterial species that control their phenotypes by quorum sensing using a range of signalling molecules. Accordingly, the present invention provides a method of controlling a phenotype regulated by quorum sensing in bacteria comprising modulating the activation by a signalling molecule of LuxR or a homologue thereof.

TABLE 2

| | Process(es) | Signal |
|---|---|---|
| Genus | regulated | Molecule(s) |
| Agrobacterium | Conjugation | HSLs |
| Enterococcus | Conjugation, | Peptides |
| | pathogenesis | |
| Erwinia | Plant symbiosis | HSLs |
| Lactococcus | Bacteriocin production | Peptides |
| Myxococcus | Development | Peptides, |
| | | amino acids |
| Pseudomonas | Mammalian | HSLs |
| | pathogenesis | |
| Ralstonia | Plant pathogenesis | HSLs |
| Staphylococcus | Pathogenesis | Peptides |
| Streptococcus | Competence, virulence | Peptides |
| Streptomyces | Antibiotic production | γ- |
| | | Butyrolactone |
| Vibrio | Bioluminescence | HSLs |

Antibodies

LuxR, homologues of LuxR or fragments of these polypeptides can be used to generate ligands, such as polyclonal or monoclonal antibodies, that are immunospecific for the polypeptides. Such antibodies may be employed to isolate or to identify clones expressing these polypeptides or to purify the polypeptides by affinity chromatography. The antibodies may also be employed as diagnostic or therapeutic aids, amongst other applications, as will be apparent to the skilled reader.

The term “immunospecific” means that the antibodies have greater affinity for the polypeptides used in the invention (namely LuxR, homologues of LuxR and fragments thereof) than for other polypeptides found on the bacterial membrane. As used herein, the term “antibody” refers to intact molecules, such as IgG, as well as to fragments thereof, such as Fab, F(ab′)2 and Fv, which are capable of binding to the antigenic determinant in question. Such antibodies thus bind to the polypeptides of the first aspect of the invention.

If polyclonal antibodies are desired, a selected mammal, such as a mouse, rabbit, goat or horse, may be immunised with a polypeptide of the method of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides used in the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985)).

Panels of monoclonal antibodies produced against the LuxR and homologous polypeptides can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a preferred example, fragments of LuxR or homologues of LuxR may be chosen and used as the antigen against which antibodies are raised. Obviously there are areas of high sequence identity between these homologues and to avoid the likelihood of cross-reactivity of antibodies, areas of low homology should be used as the antigen. This allows the production of antibodies that are specific to a particular organism and therefore treatments involving the use of such antibodies would not affect desired commensal organisms. In the present invention it has been discovered that the region between amino acids 19 and 31 is of low homology and this has been chosen for use in raising antibodies that are immunospecific for LuxR.

Anti-idiotypic antibodies may be raised, using methods known in the art, against the antibodies of the invention that immunoreact with LasR. These may bind to signalling molecules and sequester these molecules, thus preventing them from binding and activating LuxR or a homologue of LuxR.

Detection Tests

Antibodies generated by the above techniques, whether polyclonal or monoclonal, have additional utility in that they may be employed as reagents in immunoassays, radioimmunoassays (RIA) or enzyme-linked immunosorbent assays (ELISA) for the detection of bacteria utilising quorum sensing or signalling molecules. In these applications, the antibodies can be labelled with an analytically-detectable reagent such as a radioisotope, a fluorescent molecule or an enzyme.

Antibodies which specifically bind to the LuxR protein, homologues and fragments thereof may be used for the diagnosis of conditions or diseases characterised by expression of the polypeptide, or in assays to monitor patients being treated with the polypeptides, nucleic acid molecules, ligands and other compounds. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above. The invention also provides a diagnostic assay in kit form.

The signal molecule involved in quorum sensing, which in many cases is AHL, may be conjugated with a label such as a radioisotope, a fluorescent molecule or an enzyme. This may then be used in assays to detect the presence of cells that use quorum sensing.

A further type of detection test involves the screening for antibodies that are specific for LuxR or homologues thereof, and thus indicate disease. One way of doing this would be to use anti-idiotypic antibodies as mentioned above to probe for antibodies specific for LuxR or homologues thereof.

Bacteria expressing homologues of LuxR may be used to detect the presence of antibodies immunoreactive with those homologues in serum samples. Binding of antibodies to the bacteria could be detected using suitable labelled secondary antibodies.

Pharmaceutical Compositions

The invention also provides pharmaceutical compositions comprising LuxR, a homologue of LuxR, a nucleic acid encoding one of these polypeptides, fragments of these polypeptides or a ligand that binds one of these polypeptides in combination with a suitable pharmaceutical carrier. These compositions may be suitable as therapeutic or diagnostic reagents, as vaccines, or as other immunogenic compositions, as outlined in detail below.

The pharmaceutical compositions should preferably comprise a therapeutically effective amount of LuxR, a homologue of LuxR, a fragment thereof, a nucleic acid encoding one of these polypeptides or a ligand that binds one of these polypeptides. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

Gene Therapy

Gene therapy may be used to insert a nucleic acid encoding LuxR, a homologue of LuxR or a signalling molecule. This would result in production of one of these proteins which could then elicit an immune response. Such an immune response could be used to protect a host against bacterial infection. Gene therapy utilising a nucleic acid encoding LuxR, a homologue of LuxR or a fragment of those polypeptides, can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

Vaccines

Vaccines according to the invention may be prophylactic (i.e. to prevent infection) or therapeutic (i.e. to treat disease after infection). Such vaccines comprise polypeptide(s) or protein(s) according to the first aspect of the invention or nucleic acid according to the second aspect of the invention, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO/00/29428.

### BRIEF DESCRIPTION OF DRAWINGS

FIG. 1 shows an alignment of LuxR homologues. Sequences aligned using the Clustal alignment algorithm included in the LaserGene sequence analysis package. Black=invariant residues. From: Stevens, A M & Greenberg, E P (1999) Transcriptional Activation by LuxR. In: Cell-Cell Signalling in Bacteria, Eds. Dunny, G M & Winans, S C, American Society for Microbiology p 238.

FIG. 2 shows FITC labelled V. fischeri cells after pre-incubation with anti-LuxR, in A. pre-glowing; B. glowing; and C. post-glowing phases of growth.

FIG. 3 shows FITC labelled Ps. aeruginosa cells after pre-incubation with anti-LasR.

FIG. 4 shows electron microscopy images demonstrating the morphology of V. fischeri cells in pre-quorate (pre-glowing), quorate (glowing) and post-quorate (post-glowing) phases of growth.

FIG. 5 shows an AFM image demonstrating bipolar location of LuxR in V. fischeri cells

FIG. 6 shows an autoradiograph from immunoprecipitated V. fischeri cell membranes. Key to lanes: 1 & 10—Markers; 2 & 9—Empty; 3—Anti-luciferase antisera (×10 dilution); 4—Anti-luciferase antisera (undiluted); 5—Commercial anti-LuxRp antisera (×10 dilution); 6—Commercial anti-LuxRp antisera (undiluted); 7—Peptide anti-LuxRp antisera (×10 dilution); 8—Peptide anti-LuxRp antisera (undiluted).

FIG. 7 shows the changes in optical density and bioluminescence for V. fischeri cells with and without pre-incubation with commercial anti-LuxR antisera.

FIG. 8 shows changes in bioluminescence for V. fischeri cells with and without pre-incubation with the in-house prepared anti-LuxR antisera.

### MODES FOR CARRYING OUT THE INVENTION

LuxR has been previously described as being found on the inner leaflet of the bacterial cyoplasmic membrane. It is now postulated that it is only found here during certain phases of growth. Furthermore, the present invention shows that LuxR it is found on the outer surface of the outer membrane in pre-quorate and quorate cells. The signalling molecule (in the case of LuxR, N-acylated homoserine lactone) is able to bind to the receptor LuxR. It is believed that once the signalling molecule has bound, the complex is internalised and transcription is initiated.

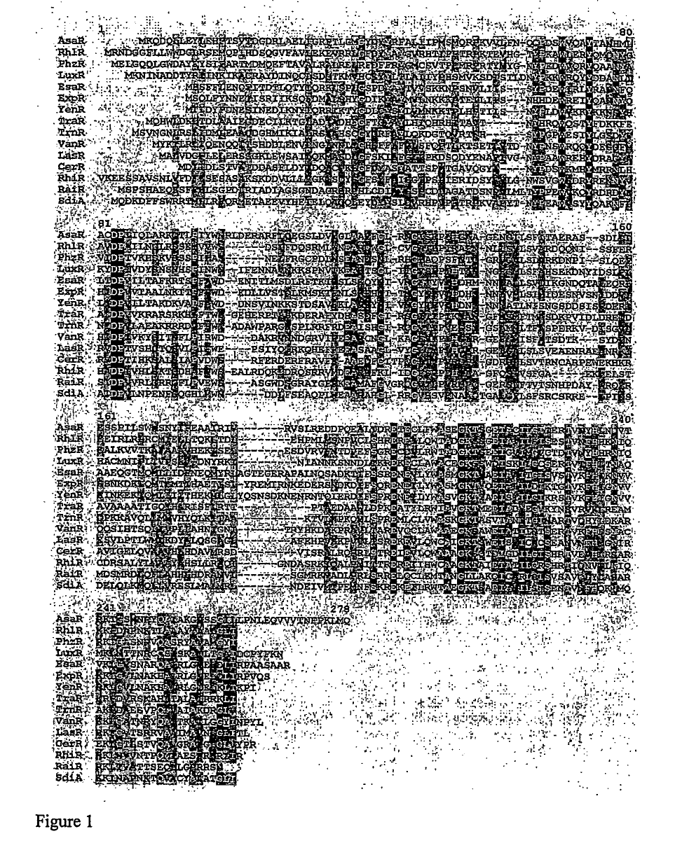

As FIG. 1 shows, there are a number of homologues of LuxR. It is predicted that, during the pre-quorate and quorate phases, these homologues are also found on the outer surface of the outer membrane. As Table 3 shows, a number of bacterial species use N-acylated homoserine lactones as a quorum sensing signalling molecule.

TABLE 3
Bacteria that produce N-acylated homoserine lactones

Agrobacterium spp.
Rhizobium spp.
Rhodobacter sphaeroides
Burkholderia cepacia
Chromobacterium violaceum
Nitrosomonas europaea
Ralstonia solanacearum
Aeromonas spp.
Citrobacter spp.
Enterobacter spp.
Erwinia spp.
Hafnia spp.
Obesumbacterium spp.
Pseudomonas aeruginosa
Pseudomonas aureofaciens
Pseudomonas fluorescens
Pseudomonas syringae
Pseudomonas putida
Rahnella aquatilis
Serratia spp.
Proteus spp.
Vibrio fischeri
Vibrio anguillarum
Vibrio harveyi
Vibrio fluvialis
Vibrio logei
Vibrio metschnikovii
Xenorhabdus nematophilus
Yersinia spp.

### EXAMPLE 1

### Identification of the Location of LuxR and its Homologues by FITC Labelling

Vibrio fischeri NRRL B-11177 (ATCC 7744) cells were grown at 25° C. in 50 ml volume nutrient broth (Oxoid)+2% NaCl in 250 ml flasks in an orbital shaker set at 200 rpm. Pseudomonas aeruginosa NCIMB 10548 was used in the experiments.

A commercially available polyclonal anti-luxR was obtained from Quorum Sciences Inc., Iowa Polyclonal anti-lasR was kindly provided by Dr. Steven Diggle, University of Nottingham.

1 ml samples of cells at the appropriate stage of the cell cycle (pre-quorate, quorate and post quorate) were aliquoted into 1.5 ml capacity microfuge tubes and centrifuged at 10,000 rpm for 3 minutes. Supernatants were discarded and the cell pellets re-suspended in 1 ml of 1×PBS. Cells were then washed twice more in 1 ml 1×PBS before final re-suspension in 500 μl 1×PBS. 25 μl of the appropriate antibody (1:1000 dilution) was then added to samples and cells incubated at 25° C. for 1 hour. Cells were washed three times in 1×PBS and then re-suspended in a final volume of 500 μl 1×PBS.

25 μl of an anti-rabbit FITC labeled secondary antibody (1:1000 dilution) was then added to test samples and cells were incubated at 25° C. for a further 1 hour. Finally, cells were washed three times in 1×PBS to remove all unbound antibody and re-suspended in a 500 μl volume of 1×PBS. Control samples were set up without antibody addition and also with the addition of the secondary antibody or primary antibody only.

For microscopic observation, a small drop of the test sample was mixed with an equal volume of mowiol, a setting agent, on a plain glass slide. A coverslip was placed over the sample and allowed to stand at 4° C. for 20 minutes to allow the setting agent to take effect. Slides were visualised using a Leica TCS 4D confocal microscope with appropriate FITC fluorescence filters.

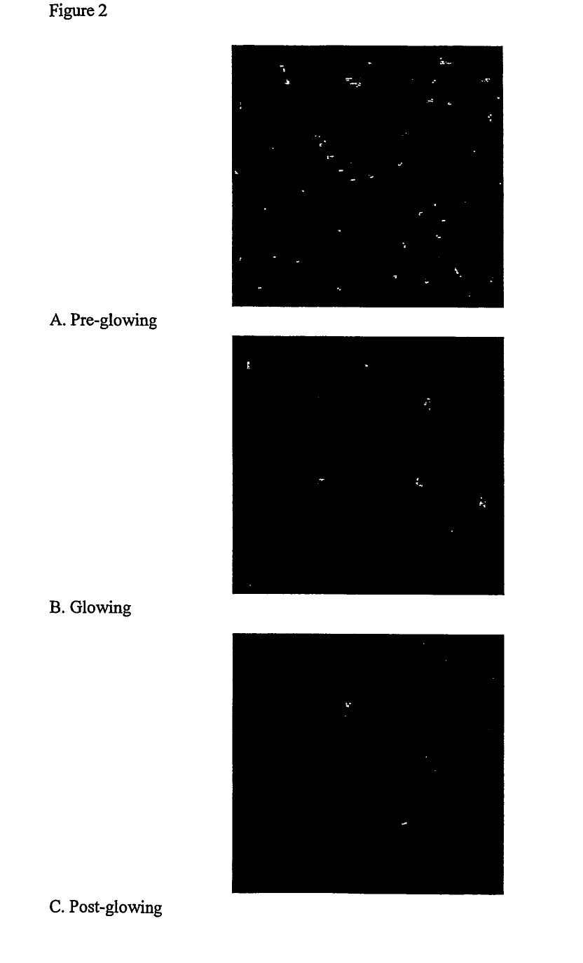

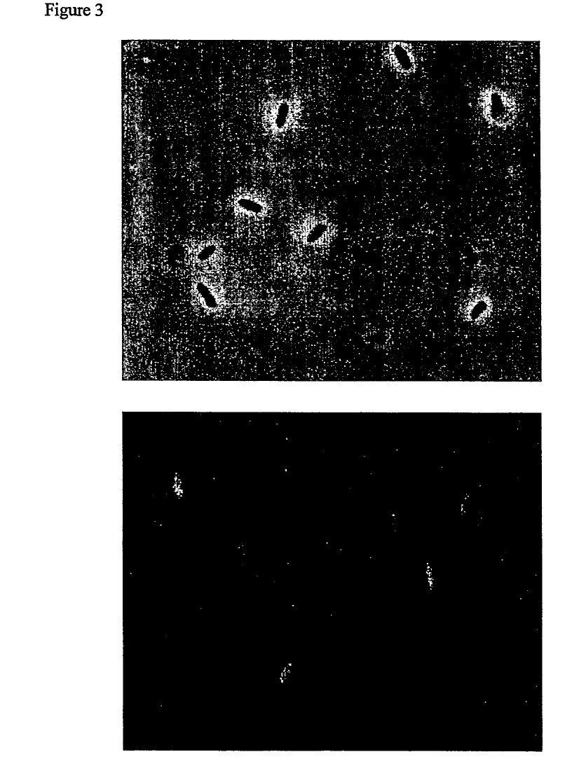

FIGS. 2 and 3 demonstrate that LuxR and LasR are found on the outer surface of the cell cytoplasmic membrane, as anti-LuxR and anti-LasR respectively are able to bind to the bacteria. The FITC labelled cells are shown as glowing spots against the dark background. In FIG. 2, there appear to be more labelled cells before the population is quorate (pre-glowing). It is postulated that LuxR and its homologues are only present on the cell membrane before quorum is reached. As quorum is reached, signalling molecules bind to LuxR and its homologues and the resultant complexes are internalised. Therefore there are few molecules of LuxR on the membrane of quorate cells, so antibody cannot bind.

### EXAMPLE 2

### Identification of the Location of LuxR and its Homologues by Gold-Labelling and Electron Microscopy

Preparation of Morphological Fixative

An 8% (v/v) stock solution of paraformaldehyde was prepared by dilution in distilled water with stirring at 60° C. 1M NaOH was added drop-wise until the solution became clear.

A 25% stock solution of glutaldehyde was prepared by dilution in distilled water. This 25% solution was then diluted 1/10 in 2×PBS to make a 2.5% solution.

The immunological fixative was prepared by combining 40 ml paraformaldehyde, 4 ml glutaldehyde, 50 ml 2×PBS and 6 ml distilled water.

Preparation of Cells

The centrifuged pellet from 1 ml of V. fischeri cell sample was placed into 2 ml of immunological fixative, re-suspended and left at room temperature for at least 2 hours.

Following fixing, the cells were washed three times in 1×PBS before re-suspension in 1 ml of 30% IMS for 1 hour at 4° C.

Cells were then centrifuged at full speed for 1 minute and re-suspended in a fresh solution of 60% IMS. The samples were then left for 1 hour at 4° C. The process was then repeated with a 90% IMS solution followed by two changes of 100% and incubated overnight in 100% IMS at −20° C.

Cells then underwent two more changes of 100% IMS, each time being left for 1 hour at −20° C. Cell samples were then centrifuged again and the IMS replaced with 1 ml of 100% de-watered ethanol and 1 ml lowiacryl resin. Samples were then placed in a freezer at −35° C. for 1 hour.

The lowiacryl/ethanol mix was then replaced with 100% lowiacryl which was then changed three times over a two-day period with samples kept at a constant temperature of −35° C.

Samples were then centrifuged at full speed for 5 minutes and pellets transferred to the bottom of gelatine capsules before being topped up with lowiacryl to eliminate most of the oxygen, which can interfere with the polymerisation, and capped.

Samples were then left at −35° C., partially covered with tin foil to prevent cracking of the samples, which can occur with direct exposure to the UV lamp required for polymerisation.

Finally the samples were placed at 25° C. under the UV lamp for a further 12 hours to complete the polymerisation process.

Samples were then cut using a diamond knife on a microtome and the sections floated onto nickel grids and dried.

Antibody Binding

The nickel grids onto which cell samples had been placed were then floated in a 50 μl droplet of anti-luxR for 1 hour at 20° C. This was followed by three washes in 1×PBS before floating in 50 μl of a gold-labeled protein A antibody for a further hour.

Grids were then washed three times in 1×PBS before being stained with uranyl acetate at 60° C. for 10 minutes, dried and examined using a Philips TEM electron microscope at magnifications of between ×9100 and ×40,000.

Atomic Force Microscopy

Preparation of Cells

1 ml cell samples were centrifuged at 10,000 rpm for 3 minutes and the pellet then re-suspended in 500 μl 1×PBS. Cells were then incubated with 25 μl of anti-luxR for 1 hour at room temperature. Samples were then washed three times in 500 μl of 1×PBS before the addition of 25 μl of protein A labeled with 10 nm diameter gold particles. Cells were again incubated for 1 hour at room temperature in the presence of the protein A and then washed three times in 1×PBS.

Preparation of Fixed Cell Samples

In order to visualize the cells a small drop of the appropriate sample was placed in the middle of a 1 cm diameter glass coverslip which had been previously glued to a 1 cm steel disc for which the AFM has a magnetic receptor. The drop of sample was left to settle for 1 hour and then any remaining liquid was blotted off with tissue. Discs were then studied using the AFM.

AFM Parameters

The atomic force microscope was used in the “tapping mode” which allowed us to get very high-resolution images of the surface of individual cells with minimal risk of damage to the cell sample. The tapping probe was set up with a 20 μm sweep such that a sizeable section of the sample could be overviewed initially. When a cell of particular interest was seen the sweep was reduced to 5 μm over the area of interest to give better resolution.

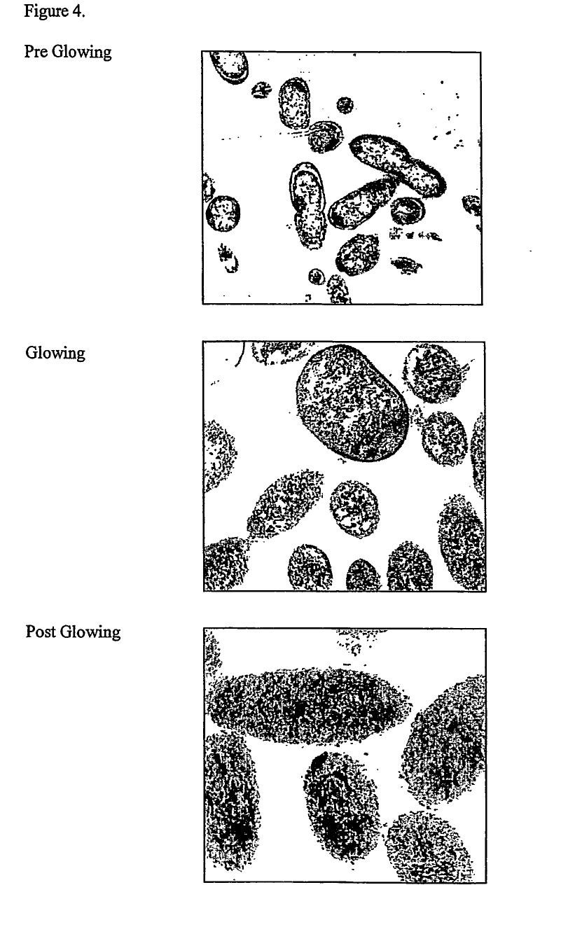

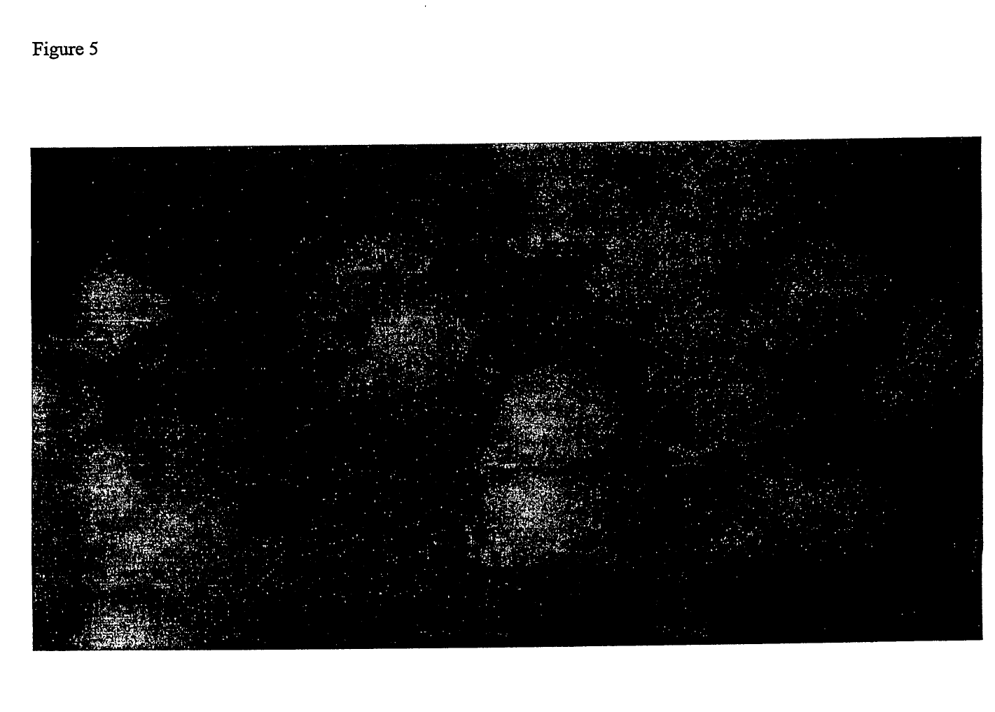

FIG. 4 shows the change in morphology of V. fischeri cells at the following stages of the cell cycle, pre-glowing (pre-quorate), glowing (quorate) and post-glowing (post-quorate). FIG. 5 is an AFM picture showing the polar binding of anti-LuxR. Clusters of the bound gold-conjugated antibody can be seen at the two poles of the bacteria. FIG. 4 also shows the reaction with antisera.

### EXAMPLE 3

### Identification of the Location of LuxR and its Homologues By Immunoprecipitation

Cultures of V. fischeri were grown in nutrient broth with 2% NaCl added. 200 nM AHL was included in the medium, prepared as 100 ml volume in 250 ml flasks.

Cultures were grown at 26° C. and 20 ml aliquots taken at the pre-quorate (OD<0.15 at 595 nm), quorate (OD<0.4 at 595 nm) and post-quorate (OD>0.5 nm at 595 nm) stages. Cells were labelled for 1 hour with 10 μCi [³⁵S] methionine ml⁻¹.

Labelled cells were then harvested by centrifugation at 3500 rpm for 5 mins at room temp.

[³⁵S] methionine labelled cells were re-suspended in 4 ml of 10 mM Tris buffer (pH 7.5 at 4° C.) containing 1 mM MgCl₂ and 1 μg DNAse I per ml. Lysozyme (50 mg/ml in 0.1M EDTA, pH 8) was added to a final conc of 50 μg ml⁻¹.

Cells were incubated on ice for 30 minutes and then broken by sonication on ice at 14 μm for 90 seconds. Remaining whole cells were removed by centrifugation at 1000 rpm for 2 minutes at 4° C. The membranes and soluble components were separated by ultracentrifugation at 100,000×g for 45 minutes at 4° C. Membranes were then suspended in 1 ml of cold 25% sucrose (w/w) ready for density gradient centrifugation.

For sucrose density gradient centrifugation, step gradients were prepared by layering 2.1 ml each of 50, 45, 40, 35, and 30% sucrose. 1 ml of membrane suspension (containing up to 8 mg of protein) was layered on top of the gradient and centrifugation carried out at 35,000 rpm for 16 hours at 4° C. 1 ml fractions were then drawn off into eppendorf tubes and fractions of interest were found by taking 100 μl samples of each and mixing with 3 ml of Optiphase “Hi-Safe-3”™ scintillation fluid (Wallac/Perkin Elmer). Disintegrations per minute (DPM) were recorded over a 60 second period using a Wallac 1410 liquid scintillation counter and those fractions showing significantly high DPM values were selected for immunoprecipitation treatment.

Samples for immunoprecipitation were treated with 1% SDS in a boiling heating block for 2 minutes followed by 50% dilution in triton buffer (0.1% triton-100 in ddH₂O pH 7.0). Non-specific precipitates were then removed by centrifugation at 6000 rpm for 3 minutes. Supernatants were then mixed with 20 μl of a 1:1000 dilution of antiserum (anti-luxR 1, anti-luxR 2, or anti-luciferase). To this mixture was added 500 μg ml⁻¹ of Staphylococcus protein A.

The tubes were then incubated on ice for 20 minutes to allow formation of antibody-protein-A complexes. The complexes were then pelleted out via centrifugation for 2 minutes at 13 000 rpm and 4° C.

Complexes were washed twice in 50 mM Tris buffer (pH 8) before being boiled for 5 minutes in 100 μl of a 1% SDS sample buffer. Proteins in the supernatants were then separated using standard SDS gel electrophoresis techniques. Gels were stained by immersing in Coomassie blue overnight, followed by destaining until bands were resolved. Gels were then washed thoroughly by immersing in ddH₂O for 1 hour then ddH₂O+3% glycerol to prevent cracking of the gel on drying. Gels were then dried at 75° C. for 2 hours using a Biorad 543 gel dryer set on the slow increase temperature ramp cycle to minimise curling of the gel.

Gels were exposed to Amersham Pharmacia Hyperfilm™ MP autoradiography film. The film was pre-flashed under darkroom conditions using an Amersham “Sensitize™” flash gun which was held 75 cm from the film and gave an optical density of 0.15 at 545 nm upon test processing samples (the ideal being 0.1-0.2 at 545 nm). This process hypersensitised the film prior to exposure to the radio-labelled gels. Gels were then placed into a film cassette and overlaid with the pre-flashed film. The cassette was sealed to ensure total evacuation of light and then placed at −80° C. for 10 days. Film was then developed using a Kodak XP-4 film processor.

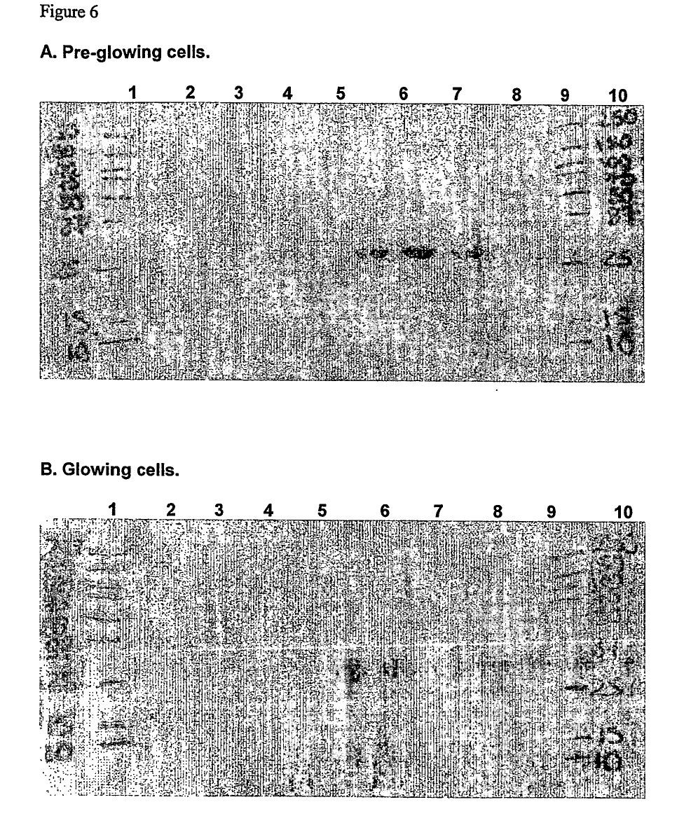

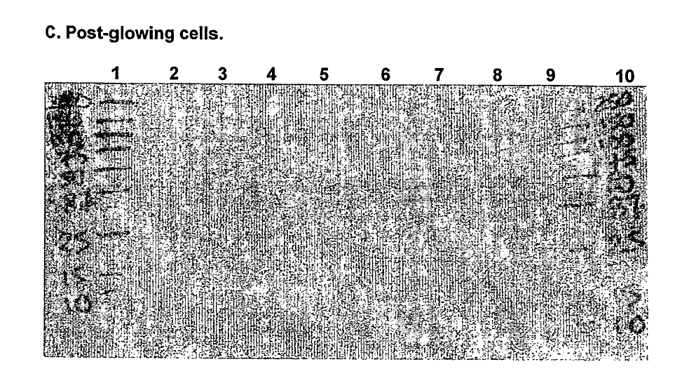

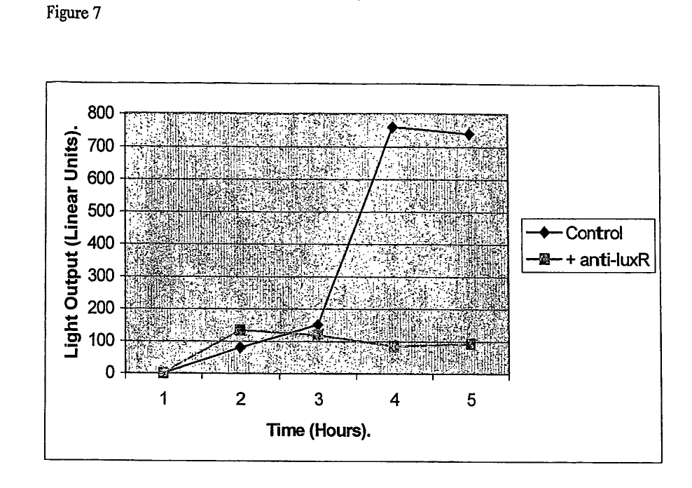

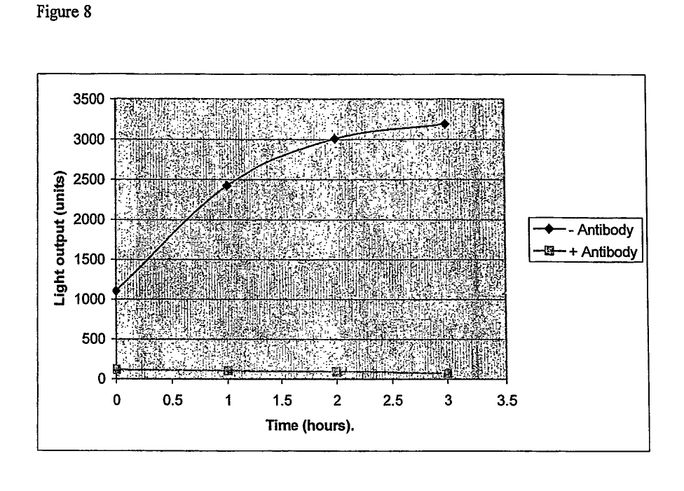

The results of the immunoprecipitation are shown in the autoradiographs in FIG. 6. In 6A, lanes 5, 6 and 7, where anti-LuxR antisera was used as a probe, clearly show bands, indicating that LuxR was present in the membrane. In 6B, the bands are less distinct, indicating a lower number of LuxR molecules in the membrane. The lack of bands in 6C shows that post-quorate cells do not have detectable amounts of LuxR in the membrane.

### EXAMPLE 4

### Production of Polyclonal Antibody

Production of Synthetic Peptide

A short synthetic peptide was produced on site in the Protein Science Facility according to the methods of Fields et al. (1990). The peptide sequence was chosen from a region close to the N-terminus of the luxR protein of V. fischeri strain ES114 (ATCC 700601). The sequence is shown below.

N-terminal residue T C N N N K D I N Q C C-
terminal residue

The chosen sequence was located between amino acids 20-30 inclusive from the N-terminus of luxR to avoid the region of the protein responsible for negative autoregulation.

Activated Keyhole Lymphocyte Haemocytoma (KLH) Preparation

0.5 ml (30 mg) of KLH (Cal Biochem) was dialysed overnight in 1 litre 50 mM Phosphate buffer, pH 7.5, 4° C. 8 mg of m-Maleimidobenzoyl-N-hydroxysuccinamide ester (MBS) dissolved in dry dimethyformamide (DMF) was added dropwise to the stirred KLH solution at room temperature and a precipitate was seen to form. The reaction mix was stirred in a stoppered vial for 30 minutes then 100 μl glycyl glycine (50 mg/ml in phosphate adjusted to pH7 with 1M NaOH) was added. The solution was the centrifuged at 13,000 rpm for 10 minutes resulting in a clear greyish solution.

The solution of activated KLH was then desalted over a Pharmacia fast desalting column equilibrated and eluted with 50 mM phosphate buffer pH7.5. The protein concentration was then determined using a standard Bradford assay and stored as 34mg aliquots at −80° C.

The synthetic peptide was then coupled to KLH using the procedure according to McCray et al. (1989). 3 mg of peptide were dissolved in 100 μl 50 mM phosphate buffer and the pH adjusted to 7.0 using 400 mM phosphate buffer pH 7.4. Half of the peptide solution was mixed with an aliquot of activated KLH and stirred in the dark for 3 hours. This mix was then dialysed into 1 litre of 1×PBS overnight. The solution was the centrifuged at 13,000 rpm for 10 minutes and desalted on a Pharmacia rapid desalting column equilibrated and eluted with 1×PBS. The protein concentration was determined using a standard Bradford assay and aliquoted as 400 μg aliquots, the equivalent of 1 rabbit immunogen dose.

Immunisation of the Rabbit

For the primary immunisation, 400 μg of the KLH/peptide conjugate was combined with a small volume (˜100 μl) of Freunds Complete Adjuvant (FCA) and administered. Two further boosters were administered at two weekly intervals. A test bleed was taken after the third injection and purified using an affinity purification technique. Dependant on the titre of antibody at that stage a further two boosters could be administered prior to the terminal bleed.

Purification

3 mg of the luxR peptide was dissolved in 2 ml of coupling buffer (50 mM Tris-HCl pH 8.5 containing 5 mM EDTA). 3 ml of “Sulfolink” coupling gel (Pierce) were transferred to a Biorad Econo-column® and washed with 12 ml of coupling buffer. The peptide solution was applied to the column and then the column stoppered and mixed end over end at room temperature for 15 minutes. The column was then allowed to stand at room temperature for 30 minutes. The column was then drained and washed with 15 ml of coupling buffer. 1.5 ml of blocking agent (30 μl β-mercaptoethanol in 5 ml coupling buffer) was added to the column and then mixed end over end for 15 minutes followed by standing for 30 minutes at room temperature. Finally the column was drained and washed with 15 ml 1M NaCl followed by 15 PBS+0.02% sodium azide.

2 μl 5 mg/ml leupeptin, 2 μl 1 mg/ml pepstatin in EtOH and 2 μl 100 mM PMSF in isopropanol were added to 4 ml of serum to inhibit proteolysis.

The serum was then applied to an appropriate affinity column equilibrated with PBS at a rate of 15 ml/hr. The flowthrough was collected for assay of possible unbound antibody. The flow rate was then increased to 30 ml/hr and washed with 10 column volumes of PBS followed by 10 column volumes of PBS containing 1M NaCl.

The antibody was then eluted with 0.1M glycine pH 2.8, containing 0.15M NaCl collecting 15×2 ml fractions. The acidic fractions were neutralised as soon as possible with 2M Tris, testing 2 μl aliquots on pH paper.

The column was also quickly neutralised with 0.1M sodium phosphate pH 7.4 and then washed with PBS containing 0.02% sodium azide for storage.

A dot blot was then carried out using a 3′3-Diaminobenzidine (DAB) stain to locate relevant fractions by probing with a peroxidase conjugated secondary antibody.

For storage the antibody containing fractions were made 1% with respect to bovine serum albumin and dialysed overnight against PBS. After dialysis 0.02% sodium azide was added and then the antibody stored as 500 μl aliquots at −20° C.

### EXAMPLE 5

### Monitoring Light Output of Cells Using Luminometry

1 ml aliquots of the culture samples, grown in luminescence media consisting of 5% yeast extract, 5% tryptone peptone, 1% CaCO₃ and 3% glycerol in filtered seawater, were placed into cylindrical, flat-bottomed cuvettes. No washing of the cells was required and light output was measured using a BioOrbit 1253 luminometer connected to a computer running the Lumicom™ data processing software. Light output was measured on a linear arbitrary scale, assuming zero to be complete darkness. Each reading was performed in triplicate and averaged.

For phenotype blocking using the commercial or in-house prepared polyclonal anti-LuxR, 1 ml samples of pre-quorate (Abs. ˜0.2 at 595 nm) V. fischeri cells were inoculated into 50 ml volume of sterile NB+2% NaCl. 25 Ξl of a 1:100 dilution of the appropriate antibody was then added to flasks. Cultures were incubated at 25° C. with shaking for 1 hour before the addition of 25 μl of a 5 mg/ml solution of synthetic AHL (2.5 μg ml⁻¹). Absorbance (595 nm) and light output of cultures recorded immediately following AHL addition and at hourly intervals thereafter.

It will be understood that the invention has been described by way of example only and modifications may be made whilst remaining within the scope and spirit of the invention.

1. A method of regulating quorum sensing in bacteria comprising modulating the activation by a signalling molecule of LuxR or a homologue thereof.

2. A method according to claim 1 wherein activation of LuxR or a homologue of LuxR by a signalling molecule is prevented.

3. A method according to claim 1 or claim 2 wherein said bacteria are Gram negative.

4. A method according any of claims 1 to 3 wherein said homologue of LuxR is selected from the list consisting of AhlR, AhyR, AsaR, BafR, BisR, BpsR, BviR, CarR, CepR, CerR, CinR, CsaR, CviR, EagR, EcbR, EchR, EsaR, ExpR, HalR, LasR, LuxS, M18752, MupR, PcoR, PhzR, PmlR, PpuR, PsmR, PsyR, RaiR, RhiR, RhlR, SdiA, SdiR, SmaR, SolR, SpnR, SprR, SwrR, TraR TriR TrlR, TrnR, VanR, VsmR, Y4qH, YenR, YpeR, YpsR, YruR, YtbR and YukR.

5. A method according to any of claims 1 to 4 wherein the signalling molecule is a N-acylated homoserine lactone.

6. A method according to any one of claims 1 to 5 wherein the binding of a ligand prevents the LuxR or homologue of LuxR from being activated by its signalling molecule.

7. A method according to claim 6 wherein said ligand is an antibody.

8. A method according to claim 7 wherein said antibody is a monoclonal antibody.

9. An antibody that immunoreacts with LuxR or a homologue of LuxR between amino acid residues 19 and 80.

10. An antibody according to claim 9 wherein said antibody immunoreacts with LuxR or a homologue of LuxR between amino acid residues 19 and 31.

11. An antibody according to claim 9 or claim 10 which immunoreacts with the sequence TCNNNKDINQC.

12. An anti-idiotypic antibody that binds to an antibody according to any one of claims 9 to 11.

13. An antibody according to any of claims 9 to 12 conjugated to a detectable label.

14. An antibody according to claim 13 wherein said label is a radioisotope, a fluorescent molecule, a heavy metal molecule or an enzyme.

15. A pharmaceutical composition comprising LuxR, a homologue of LuxR, a fragment of LuxR, a fragment of a homologue of LuxR or a nucleic acid encoding one of these polypeptides, or an antibody according to any one of claims 9 to 14.

16. A vaccine composition comprising LuxR, a homologue of LuxR, a fragment of LuxR, a fragment of a homologue of LuxR, a nucleic acid encoding one of these molecules or a quorum sensing signalling molecule.

17. A vaccine composition according to claim 16 wherein said quorum sensing signalling molecule is a N-acylated homoserine lactone.

18. A vaccine composition according to claim 16 or claim 17 further comprising a pharmaceutically acceptable diluent or carrier.

19. A vaccine composition according to any one of claims 16 to 18, further comprising an adjuvant.

20. LuxR, a homologue of LuxR, a fragment of LuxR or a homologue of LuxR, a nucleic acid encoding one of these polypeptides, an antibody according to any one of claims 9 to 14, a pharmaceutical composition according to claim 15, or a vaccine composition according to claims 16 to 19 for use as a medicament.

21. Use of LuxR, a homologue of LuxR, a fragment of LuxR or a homologue of LuxR, a nucleic acid encoding one of these polypeptides, an antibody according to any one of claims 9 to 14, a pharmaceutical composition according to claim 15, or a vaccine composition according to claims 16 to 19 in the manufacture of a medicament for the treatment of disease in which quorum sensing is implicated.

22. Use of LuxR, a homologue of LuxR, a fragment of LuxR or a homologue of LuxR, a nucleic acid encoding one of these polypeptides, a ligand, an antibody according to any one of claims 9 to 14, a pharmaceutical composition according to claim 15, or a vaccine composition according to claims 16 to 19 for sensitising an antibiotic resistant bacterium to an antibiotic.

23. Use of LuxR, a homologue of LuxR, a fragment of LuxR or a homologue of LuxR, a nucleic acid encoding one of these polypeptides, a ligand, an antibody according to any one of claims 9 to 14, a pharmaceutical composition according to claim 15, or a vaccine composition according to claims 16 to 19 in the manufacture of a medicament for sensitising an antibiotic resistant bacterium to an antibiotic.

24. Use of LuxR, a homologue of LuxR, a fragment of LuxR or a homologue of LuxR, a nucleic acid encoding one of these polypeptides, a ligand, an antibody according to any one of claims 9 to 14, a pharmaceutical composition according to claim 15, or a vaccine composition according to claims 16 to 19 in the manufacture of a medicament for the treatment of a disease in which quorum sensing is implicated wherein the patient suffering from that disease is refractive to antibiotic therapy.

25. Use of LuxR, a homologue of LuxR, a fragment of LuxR or a homologue of LuxR, a nucleic acid encoding one of these polypeptides, a ligand, an antibody according to any one of claims 9 to 14, a pharmaceutical composition according to claim 15, or a vaccine composition according to claims 16 to 19 in the manufacture of a medicament for the treatment of a disease in which quorum sensing is implicated wherein the medicament is administered in conjunction with an antibiotic.

26. Use of an antibiotic in the manufacture of a medicament for the treatment of a disease in which quorum sensing is implicated wherein the subject being treated is pre-administered with a pharmaceutical composition or vaccine according to the invention.

27. The use of claim 21 wherein disease is caused by Bacillus subtilis, Streptococcus pneumoniae, Staphylococcus aureas, Vibrio salmonicida, Aeromonas hydrophila, Burkhoderia ambifaria, Burkholderia pseudomallei, Burkholderia mallei, Burkholderia stabilis, Burkholderia vietnamiensis, Burkholderia multivorans, Escherichia coli, Serratia marcescens, Salmonella typhi, Brucella suis, Brucella melitensis, Yersinia ruckeri, Hafina alvei, Shigella flexneri, Serratia liquefaciens, Enterococcus faecalis, Pseudomonas aeruginosa, Burkholderia cepacia, Pseudomonas fluorescens, Providencia stuartii, Klebsiella aerogenes, Yersinia pestis, Yersinia enterocolitica or Yersinia pseudotuberculosis.

28. The use according to any one of claims 21 to 27 wherein said disease is Crohn's disease or Cystic Fibrosis, cellulites and ecthyma, Glanders, melioidosis, meningitis, septicaemia, pneumonia, enteric infections and urinary tract infections, food poisoning, chest infections, typhoid fever, Malta disease, blood stream infections, shigellosis, salmonelliosis, black death and gastroenteritis, hitra disease in Atlantic salmon, haemmorrhagic septicaemia in marine fish, spontaneous abortion in pigs and sheep, red mouth disease in rainbow trout, and cranial and eye lesions in fish.

29. The use according to any one of claims 22-26 wherein said antibiotic is erythromycin A, rifampin, tetracycline, chloramphenicol, norfloxacin, nalidixic acid or penicillin G.

30. Use of a method according to any one of claims 1 to 8 for the inhibition of biofilms.

31. A method of detection of quorum sensing bacteria comprising;
(i) probing a sample of bacteria with a labelled antibody according to claim 13 or claim 14, and
(ii) detecting the presence of antibody attached to bacteria.

32. A method of detection of quorum sensing bacteria comprising;
(i) probing a sample of bacteria with a first antibody according to any one of claims 7 to 10,
(ii) probing said first antibody with a second, labelled antibody, and
(iii) detecting the presence of the second antibody attached to bacteria.

33. A method of detecting antibodies specific for LuxR or a homologue thereof comprising:
(i) probing a sample of serum with whole bacterial cells expressing whole or a fragment of LuxR or a homologue thereof,
(ii) probing the bacteria/antibody complex with a second, labelled antibody, and
(iii) detecting the presence of the second antibody attached to the bacteria/first antibody complex.

34. A method of detecting antibodies specific for LuxR or a homologue thereof comprising:
(i) probing a sample of serum with purified LuxR or a fragment or homologue thereof,
(ii) probing the bacterial protein/antibody complex with a second, labelled antibody, and
(iii) detecting the presence of the second antibody attached to the bacteria/first antibody complex.

35. A kit comprising an antibody according to any one of claims 8 to 14.

36. A kit comprising a fragment of LuxR or a fragment of a homologue of LuxR for the detection of antibodies thereto.

37. A method of inhibiting quorum sensing comprising sequestering quorum sensing signal molecules.

38. Use of an antibody according to claim 12 in the method of claim 37.

39. A kit comprising for simultaneous, separate or sequential use (i) LuxR, a homologue of LuxR, a fragment of LuxR or a homologue of LuxR, a nucleic acid encoding one of these polypeptides, an antibody, a pharmaceutical composition or a vaccine composition according to the invention and (ii) an antibiotic.
